(19) United States
(12) Patent Application Publication
(10) Pub. No.: US 2006/0171905 A1
(43) Pub. Date: Aug. 3, 2006

(54) Dental bleaching compositions having a protective coating applied thereto

(76) Inventor: Peter M. Allred, Riverton, UT (US)

Correspondence Address: Rick D. Nydegger;WORKMAN NYDEGGER, 1000 Eagle Gate Tower, 60 East South Temple, Salt Lake City, UT, 84111, US

(21) Appl. No.: 11/047,051

(22) Filed: Jan. 31, 2005

(51) Int. Cl. A61K 8/22

(52) U.S. Cl. 424/53

(57) ABSTRACT

A dental bleaching material comprises a dental bleaching composition that includes a dental bleaching agent and a thickening agent and a protective coating on a surface of the dental bleaching composition that includes a complex of polyvinyl pyrrolidone (PVP) and hydrogen peroxide (H₂O₂). The bleaching composition may be a gel or solid. The protective coating may be a powder or a solid shell. A dental bleaching device includes a barrier layer, such as an initially flat strip or a dental bleaching tray, a dental bleaching composition adjacent to the barrier layer, and a protective coating on a surface of the dental bleaching composition comprising the complex of PVP and H₂O₂.

### BACKGROUND OF THE INVENTION

1. The Field of the Invention

The present invention is in the field of dental treatment materials. More particularly, the invention relates to bleaching compositions that can be used to bleach a patient's teeth.

2. The Relevant Technology

Virtually all people desire white or whiter teeth. To achieve this goal, people have veneers placed over their teeth or have their teeth chemically bleached. A common bleaching method involves the use of a dental tray that is custom-fitted to a person's teeth and that is therefore comfortable to wear. One type of customized tray is made from a stone cast of a person's teeth. Another is customized directly using a person's teeth as a template (e.g., “boil-and-bite” trays). Non-customized trays that approximate the shapes and sizes of a variety of users' dental arches have also been used. A dental bleaching composition is placed into the tray and the tray placed over the person's teeth for a desired period of time.

A dental bleaching composition is often supplied in a separate container (e.g., a syringe or tube) from which it can be dispensed into the dental tray prior to placing the tray over the person's teeth. Alternatively, some dental trays are provided pre-filled with a dental bleaching composition.

One disadvantage of pre-filled dental trays is that the dental bleaching compositions, if not sealed within a container, tend to dry out, forming a more solid gel material and possibly losing long-term stability. This is also true in the case of bleaching compositions pre-filled within a dental tray and sealed within a package, because of the head space between the bleaching composition and surrounding package.

It would be an improvement in the art to provide a dental bleaching material without the aforementioned difficulties.

### BRIEF SUMMARY OF THE PREFFERED EMBODIMENTS

The present invention is directed to a dental bleaching material comprising a dental bleaching composition that includes a dental bleaching agent and a thickening agent. The dental bleaching material also includes a protective coating on a surface of the dental bleaching composition that includes a complex of polyvinyl pyrrolidone and hydrogen peroxide. The dental bleaching composition may comprise a gel or solid.

According to one embodiment, the dental bleaching agent comprises at least one of aqueous hydrogen peroxide, carbamide peroxide, metal perborates, metal percarbonates, metal peroxides, metal chlorites and hypochlorites, peroxy acids, and peroxy acid salts.

According to one embodiment, the dental bleaching agent has a concentration in a range of about 5% to about 80% by weight, more preferably in a range of about 10% to about 60% by weight, and most preferably in a range of about 20% to about 50% by weight.

According to one embodiment the thickening agent comprises at least one of polyvinyl pyrrolidone (PVP), PVP-vinyl acetate copolymers, carboxypolymethylene, polyethylene oxide, polyacrylic acid, polyacrylic acid polymers or copolymers, polyacrylates, polyacrylamides, copolymers of polyacrylic acid and polyacrylamide, carboxymethylcellulose, carboxypropylcellulose, cellulosic ethers, polysaccharide gums, and protein.

According to one embodiment, the dental bleaching composition may include additional active agents. Examples of additional active agents include desensitizing agents, remineralizing agents, antimicrobial agents, anti-plaque agents, anti-tartar agents, gingival soothing agents, anesthetics, anti-oxidants, and mouth freshening agents.

The protective coating may comprise a dry powder that adheres to the surface of the dental bleaching composition. A dry powder protective coating may be applied by dusting the dental bleaching composition with the dry powder.

Alternatively, the protective coating may comprise a soft shell that adheres to the dental bleaching composition surface. A soft shell protective coating may be formed by first dissolving a polyvinyl pyrrolidone/hydrogen peroxide complex in a suitable solvent (e.g., water, ethanol, glycerol, various glycols, or any other suitable solvent into which PVP dissolves), and then applying the solution to the dental bleaching composition surface by spraying, dipping, spreading, or any other suitable method of application. Once the solution dries, a soft shell of protective coating forms over the surface of the dental bleaching composition.

An associated inventive dental bleaching device includes a moisture resistant barrier layer, a dental bleaching composition including a dental bleaching agent and a thickening agent adjacent to the barrier layer, and a protective coating on a surface of the dental bleaching composition that includes a complex of polyvinyl pyrrolidone and hydrogen peroxide.

According to one embodiment, the dental bleaching composition may comprise a bead of dental bleaching gel applied adjacent to the barrier layer. The bead may have any desired cross-sectional diameter, and according to one embodiment the bead includes a cross-sectional diameter between about 1 mm and about 5 mm, and more preferably between about 2 mm and about 4 mm.

Alternatively, the dental bleaching composition may comprise a substantially solid dental bleaching composition, which may be applied as a continuous layer on an inner surface of the barrier layer. When the dental bleaching composition is applied as a continuous layer on the barrier layer it may have any desired thickness. Preferably the continuous layer has a thickness in a range of about 0.25 mm to about 2 mm, and more preferably in a range of about 0.5 mm to about 1 mm.

According to one embodiment, the protective coating may be applied so as to cover at least a portion of the exposed surface of the dental bleaching composition. Alternatively, the protective coating may be applied so as to also cover at least a portion of the moisture resistant barrier layer.

Even though the inventive dental bleaching device includes a protective coating, according to some embodiments, it may also be desirable for the device to be contained within a sealed package.

According to one embodiment the dental bleaching device may be manufactured by providing a moisture resistant barrier layer, applying a dental bleaching composition that includes a dental bleaching agent and a thickening agent so as to be adjacent to the barrier layer, and applying a protective coating on a surface of the dental bleaching composition, the protective coating including a complex of polyvinyl pyrrolidone and hydrogen peroxide.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a perspective view of an exemplary tray-shaped bleaching device including a barrier layer, a dental bleaching composition applied as a bead on an inner surface of the barrier layer, and a protective coating applied over the dental bleaching composition;

FIG. 1B is a perspective view of an exemplary tray-shaped bleaching device including a barrier layer, dental bleaching composition applied as a continuous layer on an inner surface of the barrier layer, and a protective coating applied over the dental bleaching composition;

FIG. 2 is a perspective view of an exemplary tray-shaped bleaching device including a barrier layer having an L-shaped cross section, a dental bleaching composition applied as a bead on an inner surface of the barrier layer, and a protective coating applied over the dental bleaching composition;

FIG. 3 is a perspective view of an exemplary dental bleaching device including a barrier layer having a U-shaped trough, a continuous layer of a dental bleaching composition within the trough, and a protective coating applied over the dental bleaching composition;

FIG. 4 is a perspective view of an exemplary dental bleaching device including a barrier layer having a V-shaped trough, a dental bleaching composition in the form of a continuous layer within the trough, and a protective coating applied over the dental bleaching composition;

FIG. 5 is a perspective view of an exemplary dental bleaching device or strip having an initially flat barrier layer, a dental bleaching composition in the form of a continuous layer on the surface of the barrier layer, and a protective coating applied over the dental bleaching composition;

FIG. 6 is a perspective view of an exemplary dental bleaching device packaged within a sealed container;

FIG. 7A illustrates a person placing a dental bleaching device according to the invention over the upper dental arch; and

FIG. 7B illustrates a person with dental bleaching devices placed over the upper and lower dental arches.

### DETAILED DESCRIPTION OF THE PREFERRED EMBODIMENTS

I. Introduction and Definitions

A detailed description of the invention will now be provided with specific reference to figures illustrating preferred embodiments of the invention. It will be appreciated that like structures will be provided with like reference designations. Certain terms used throughout the detailed description will now be defined.

The term “barrier layer”, as used herein, refers to one or more layers of a material that protects the dental bleaching composition from ambient moisture and saliva found within a person's mouth when the dental bleaching device is placed over the person's teeth. According to some embodiments, a barrier layer may be shaped. For example, it may include a front side wall and a rear side wall. According to one such specific embodiment the barrier layer may be shaped as a dental tray.

The terms “longitudinal”, “longitudinal dimension”, and “longitudinal profile”, as used herein when referring to a shaped barrier layer or dental bleaching device, shall refer to the lengthwise dimension of the barrier layer or device. The barrier layer or device may be straight in the “longitudinal dimension” or it may be horseshoe-shaped or otherwise “longitudinally curved” in the longitudinal dimension so as to approximate the curvature of a person's dental arch, or at least facilitate placement of the barrier layer or device over the dental arch.

The term “viscous gel” or “gel”, as used herein, shall refer to dental bleaching compositions that have been formulated or processed so that they do not readily flow by the force of gravity but are viscous so that they can be expressed from a syringe orifice or other dispensing means known in the art.

At some point, when the viscosity of a highly viscous gel becomes so great as to yield a composition that is substantially solid but still plastically deformable, it may be considered to be a “stiff putty”. The difference between a “viscous gel” and a “stiff putty” is a matter of degree. Likewise, a “stiff putty” can become so stiff or rigid as to become a “solid”.

The term “substantially solid”, as used herein, refers to a dental bleaching composition that is in a solid or semi-solid condition. In one aspect, a “substantially solid” composition can be characterized as a cohesive mass that does not readily flow or separate when subjected to gravitational forces and which cannot be readily expressed through a syringe outlet or other similarly-sized opening or orifice. Thus, the term “substantially solid” excludes runny liquids, viscous liquids, and even thick gels that are able to flow when subjected to gravity and/or which can be readily expressed through a syringe outlet or other similarly-sized opening or orifice. The term “substantially solid”, when used in the context of a dental bleaching composition, also excludes dry particulate compositions or powders because dry particulates and powders readily flow when subjected to gravity and/or are readily separated (i.e., the particles as a whole have little or no internal cohesion). Moreover, powders or particulates, when viewed as a whole, are not coherent or solid.

The term “molecular weight”, as used herein, shall refer to number average molecular weight expressed in Daltons, unless otherwise specified.

The present invention relates to an inventive dental bleaching material, a related dental bleaching device, and methods for using and making the same. The inventive dental bleaching material comprises a dental bleaching composition and a protective coating on a surface of the dental bleaching composition. The dental bleaching composition includes a dental bleaching agent and a thickening agent. The protective coating includes a complex of polyvinyl pyrrolidone and hydrogen peroxide.

II. Exemplary Dental Bleaching Materials

The inventive dental bleaching material comprises a dental bleaching composition that includes a dental bleaching agent and a thickening agent, and a protective coating applied over a surface of the dental bleaching composition. The protective coating includes a complex of polyvinyl pyrrolidone (PVP) and hydrogen peroxide.

Examples of dental bleaching agents that can be used within dental bleaching compositions according to the invention include, but are not limited to, aqueous hydrogen peroxide, carbamide peroxide, metal perborates (e.g., sodium perborate), metal percarbonates (e.g., sodium percarbonate), metal peroxides (e.g., calcium peroxide), metal chlorites and hypochlorites, peroxy acids (e.g., peroxyacetic acid), and peroxy acid salts.

Bleaching agents within the dental bleaching composition according to the invention can have any desired concentration, e.g., between 1-90% by weight of the dental bleaching composition. The concentration of the dental bleaching agent can be adjusted depending on the intended treatment time for each bleaching session. In general, the shorter the treatment time, the more bleaching agent will be added to accelerate dental bleaching so as to effect bleaching in a shorter time period. One or more bleaching agents are preferably included in an amount in a range of about 5% to about 80% by weight of the dental bleaching composition, more preferably in a range of about 10% to about 60% by weight, and most preferably in a range of about 20% to about 50% by weight.

Examples of other active agents that may be included in addition to the dental bleaching agent include desensitizing agents (e.g., potassium nitrate, other potassium salts, citric acid, citrates, and sodium fluoride), remineralizing agents (e.g., sodium fluoride, stannous fluoride, sodium monofluorophosphate, and other fluoride salts), antimicrobial agents and preservatives (e.g., chlorhexidine, triclosan, sodium benzoate, parabens, tetracycline, phenols, and cetyl pyridinium chloride), anti-plaque agents, anti-tartar agents (e.g., pyrophosphates), gingival soothing agents (e.g., aloe vera, mild potassium nitrate, isotonic solution-forming salts), anesthetics (e.g., benzocaine, lidocain, and the like), anti-oxidants (e.g., vitamin A, vitamin C, vitamin E, other vitamins, and carotene), and mouth freshening agents (e.g., camphor and wintergreen)

Examples of thickening agents include a wide variety of hydrophilic polymers. Examples of hydrophilic polymer thickening agents include, but are not limited to, polyvinyl pyrrolidone (PVP), PVP-vinyl acetate copolymers, carboxypolymethylene (e.g., CARBOPOL, sold by Novean, Inc.), polyethylene oxide (e.g., POLYOX, made by Union Carbide), polyacrylic acid polymers or copolymers (e.g., PEMULEN, sold by Novean, Inc.), polyacrylates, polyacrylamides, copolymers of polyacrylic acid and polyacrylamide, carboxymethylcellulose, carboxypropylcellulose, cellulosic ethers, polysaccharide gums, proteins, and the like.

Non-limiting examples of polyvinyl pyrrolidone polymers that have been used in formulating dental bleaching compositions according to the invention include Kollidon 30, a polyvinyl pyrrolidone polymer sold by BASF having a molecular weight of 50,000, Kollidon VA 60, a polyvinyl pyrrolidone polymer having a molecular weight of 60,000, and Kollidon 90 F, a polyvinyl pyrrolidone polymer having a molecular weight of 1.3 million.

When the dental bleaching composition comprises a gel, the one or more thickening agents are preferably included in an amount in a range of about 1% to about 50% by weight of the bleaching gel, more preferably in a range of about 3% to about 30% by weight, and most preferably in a range of about 5% to about 20% by weight.

When the dental bleaching composition is substantially solid, the one or more thickening agents are preferably included in an amount in a range of about 10% to about 90% by weight of the substantially solid treatment composition, more preferably in a range of about 20% to about 80% by weight, and most preferably in a range of about 40% to about 75% by weight.

The protective coating may comprise a dry powder or a soft shell. The protective coating protects the bleaching agent within the dental bleaching composition from premature activation and/or decomposition as a result of exposure to air. The polyvinyl pyrrolidone-hydrogen peroxide complex of the protective coating may comprise any suitable PVP-hydrogen peroxide complex. Examples of suitable complexes include PEROXYDONE K-30, PEROXYDONE K-90, and PEROXYDONE XL-10, all made by International Specialty Products.

According to one embodiment, the protective coating comprises between about 5% to about 30% hydrogen peroxide by weight, more preferably between about 10% to about 25% hydrogen peroxide by weight, and most preferably between about 15% to about, 22% hydrogen peroxide by weight.

The protective coating protects the dental bleaching composition prior to use. Once the protective coating contacts water or the patient's saliva, the coating dissolves, allowing the underlying dental bleaching composition to adhere to and begin bleaching the teeth. Because the protective coating itself releases hydrogen peroxide when moistened with saliva or water, it is also active in bleaching teeth to some degree.

III. Exemplary Dental Bleaching Devices

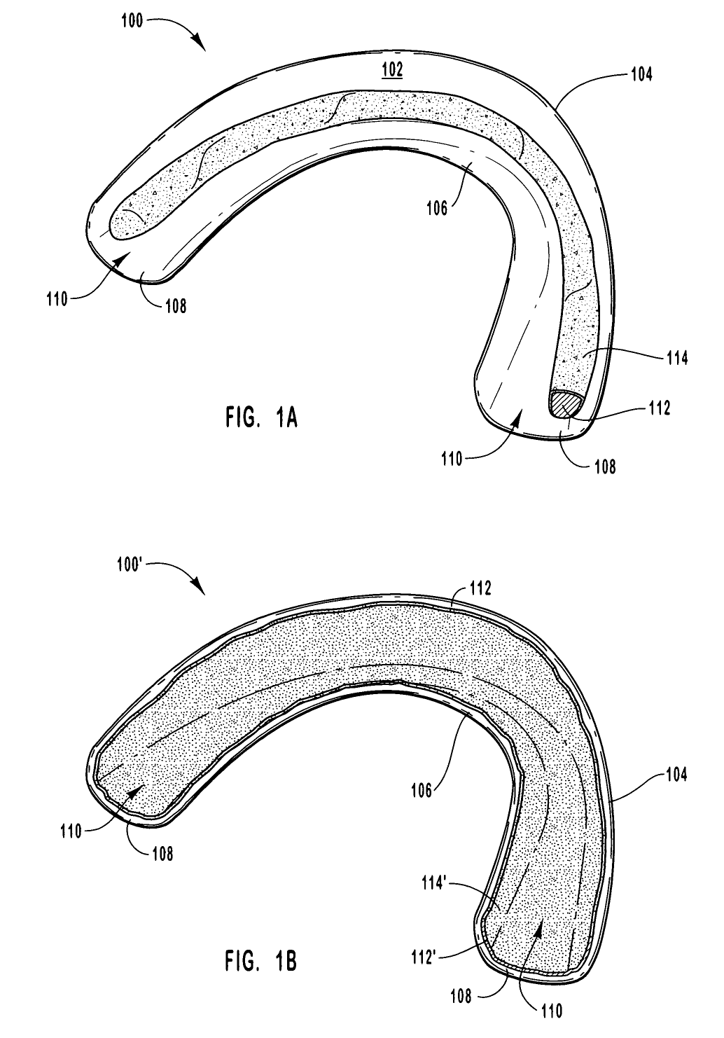

FIGS. 1A through 5 illustrate several exemplary dental bleaching devices, each including a dental bleaching composition having a protective coating.

Exemplary dental bleaching devices having a dental tray shaped barrier layer are depicted in FIGS. 1A and 1B. FIG. 1A is a perspective view of a dental bleaching device 100 comprising a moisture resistant barrier layer 102 having a front side wall 104, a rear side wall 106, and a bottom wall 108 that together define a trough 110 having a generally U-shaped cross section throughout the entire longitudinal dimension. Disposed within the trough 110 is a bead of dental bleaching gel 112. As can be seen in the cut away section of the bead of bleaching gel 112, a protective coating 114 has been applied over the otherwise exposed surface of gel 112.

Although the bead of dental bleaching gel 112 may have any desired cross sectional diameter, the bead of dental bleaching gel 112 preferably has a cross-sectional diameter between about 1 mm and about 5 mm, and more preferably between about 2 mm and about 4 mm.

FIG. 1B depicts a dental bleaching device 100′ comprising a moisture resistant barrier layer 102 having a front side wall 104, a rear side wall 106, and a bottom wall 108 that together define a trough 110 having a generally U-shaped cross section throughout the entire longitudinal dimension. Disposed within the trough 110 is a continuous layer of a substantially solid dental bleaching composition 112′. As can be seen in the cut away section of the layer of substantially solid dental bleaching composition 112′, a protective coating 114′ has been applied over the otherwise exposed surface of composition 112′.

Although the continuous layer of treatment composition 112′ may have any desired thickness, preferably the thickness is between about 0.2 mm and about 2 mm, more preferably between about 0.5 mm and about 1 mm.

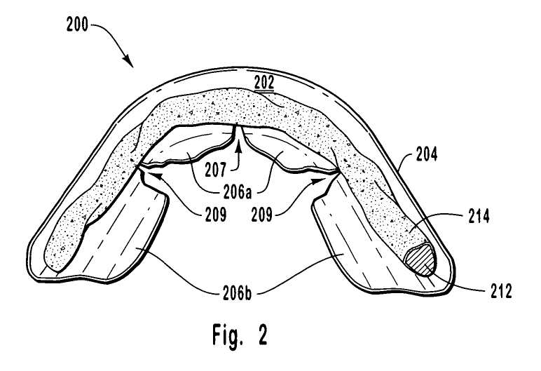

FIG. 2 illustrates an alternative dental bleaching device 200 that includes a moisture resistant barrier layer configured so as to have a noncontiguous bottom or rear wall. Instead of contiguous sidewalls, dental bleaching device 200 includes a barrier layer 202 having a front sidewall 204, first bottom flaps 206a separated by a first cut or discontinuity 207, and second bottom flaps 206b separated from the first bottom flaps 206a by second cuts or discontinuities 209. The first bottom flaps 206a are sized and configured so as to wrap around and lie adjacent to the inner surfaces of a person's incisors and canines when in use. Second bottom flaps 206b are sized and configured so as to wrap around and contact inner surfaces of a person's bicuspids and optionally one or more molars.

The cuts or discontinuities 209 between first flaps 206a and second flaps 206b facilitate good contact of the first bottom flaps 206a to a person's incisors and canines, particularly at the junction of the canines and bicuspids. The cuts or discontinuities 209 compensate for the abrupt difference in width between a person's bicuspids adjacent to the second bottom flaps 206b and the canines adjacent to the first bottom flaps 206a. The discontinuity or cut 207 separating first bottom flaps 206a from each other further assists in conforming the flaps 206a to the inner surfaces of a person's incisors and canines.

Although FIG. 2 shows a bead of dental bleaching gel 212 disposed adjacent to barrier layer 202, the dental bleaching gel 212 may alternatively be applied as a substantially continuous layer. As can be seen in the cut away section of the bead of bleaching gel 212, a protective coating 214 has been applied over the otherwise exposed surface of gel 212.

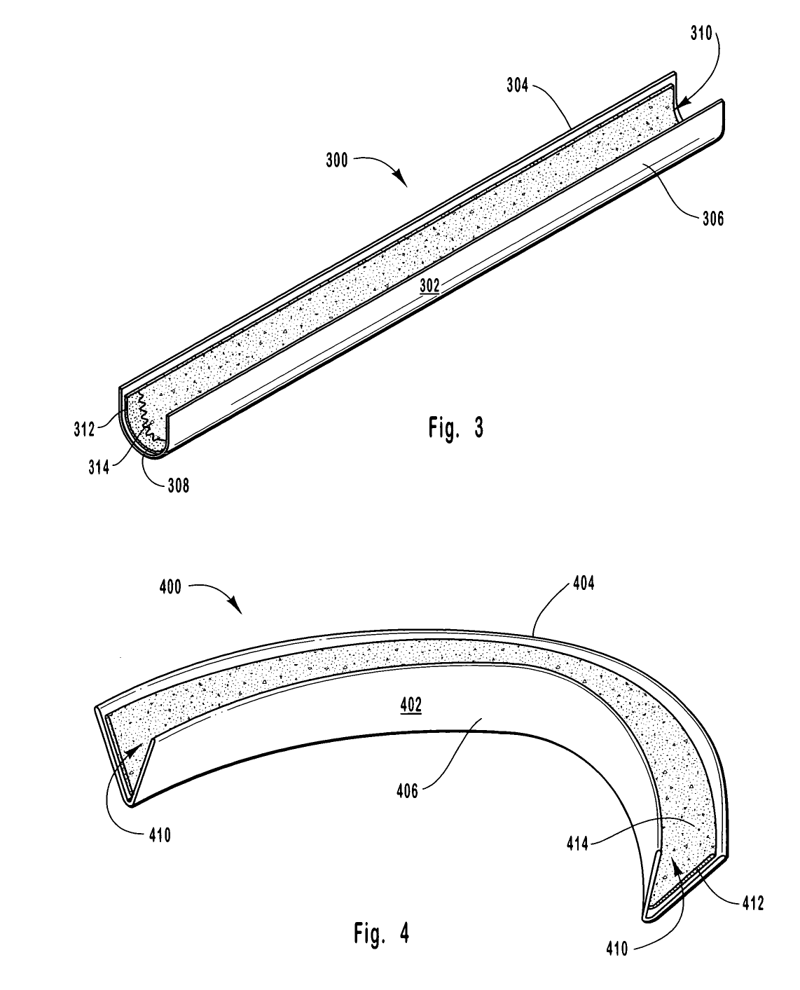

FIG. 3 depicts an alternative embodiment of a dental bleaching device 300 having a U-shaped trough and a substantially linear or straight longitudinal profile. More particularly, the dental bleaching device 300 includes a moisture resistant barrier layer 302 having a front sidewall 304, a rear sidewall 306, and a bottom sidewall 308 that together define a U-shaped trough 310 into which a continuous layer of dental bleaching composition 312 is disposed, and over which a protective coating 314 is applied. A bead of bleaching gel could alternatively replace the continuous layer of dental bleaching composition 312.

FIG. 4 depicts another alternative embodiment of a dental bleaching device 400 or having a V-shaped trough. More particularly, the dental bleaching device 400 includes a barrier layer 402 having a front sidewall 404 and a rear sidewall 406 that together define a V-shaped trough 410 within which a continuous layer of dental bleaching composition 412 is disposed, and over which a protective coating 414 is applied. A bead of bleaching gel could alternatively replace the continuous layer of dental bleaching composition 412.

FIG. 5 depicts an alternative embodiment of a dental bleaching device 500 having a flat, strip-like shape. More particularly, the dental bleaching strip 500 includes a moisture resistant barrier, layer 502 having a flat, strip-like configuration onto which a continuous layer of dental bleaching composition 512 is disposed, and over which a protective coating 514 is applied. A bead of a bleaching gel could alternatively replace the continuous layer of dental bleaching composition 512.

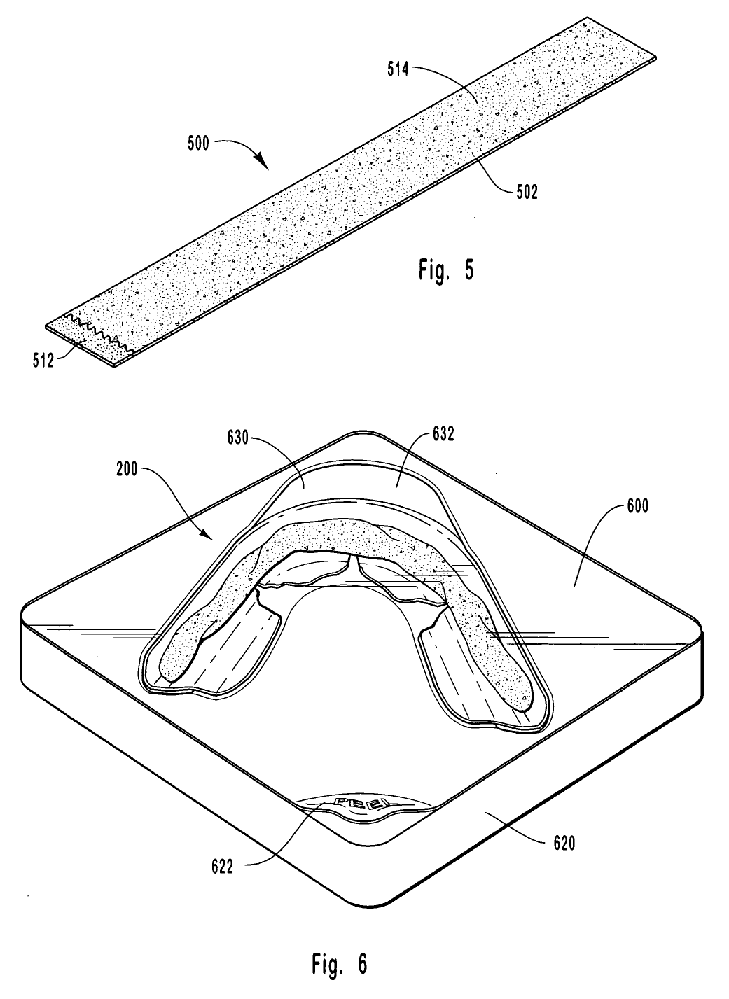

Although the inventive dental bleaching device includes a protective coating over the dental bleaching composition for protection of the composition against contaminants or loss of potency during storage and prior to use, the inventive dental bleaching devices may still be packaged within a sealed container or package prior to use. As illustrated in FIG. 6, exemplary dental bleaching device 200 can be sealed within a protective package 600 that includes a rigid support layer 620 and a peelable cover 622. When it is desired to use the dental bleaching device 200, the peelable cover 622 is removed and the bleaching device 200 is removed or separated from the support layer 620.

According to one embodiment, the dental bleaching device 200 may be packaged with a nesting exoskeleton 630. In use, both the bleaching device 200 and exoskeleton 630 can be placed into a person's mouth so as to initially position the treatment device 200 over the person's teeth and/or gums. The exoskeleton 630 may include a handle 632 to facilitate placement and positioning within the mouth. Thereafter, the exoskeleton 630 is removed, leaving only the treatment device 200 within the person's mouth. This permits further manipulation of the barrier layer 202 in order for the treatment device 200 to better conform to the shape and irregularities of the person's teeth.

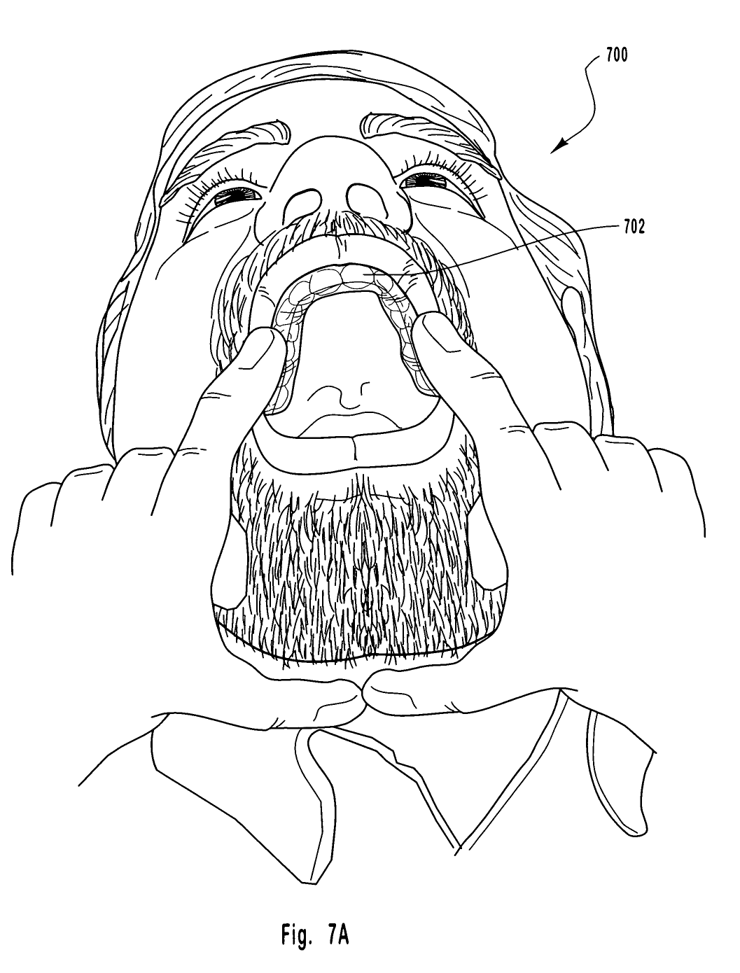

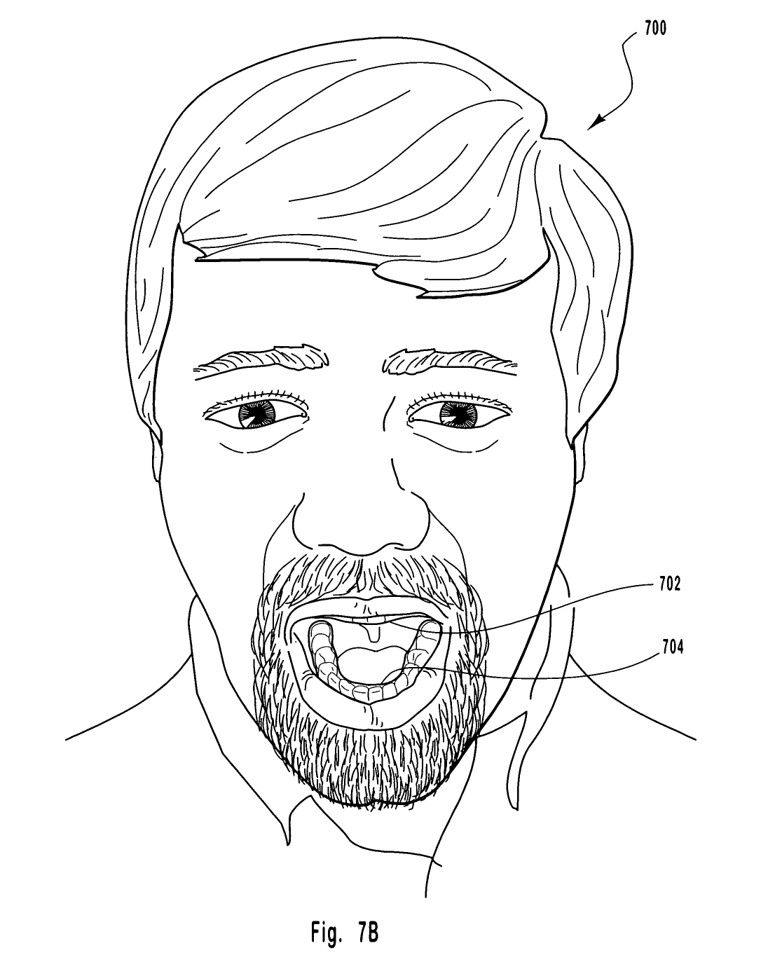

FIG. 7A illustrates a person 700 placing a dental bleaching device 702 over the person's upper dental arch. FIG. 7B shows the person 700 with both a dental bleaching device, 702 over the person's upper dental arch and a bleaching device 704 over the lower dental arch. It will be appreciated that the dental bleaching devices 702 and 704 can be placed over a person's upper and lower dental arches in any desired order.

To remove the dental bleaching device, a user can pry open a corner of the barrier layer using a fingernail or rigid tool and then pull the remainder off. Any residual dental bleaching composition that remains adhered to the person's teeth can be removed by washing or flushing water over the person's teeth, and/or by brushing. Although the inventive dental bleaching compositions can be very adhesive to teeth when protected from excessive moisture, they can be formulated to quickly break down and dissolve when flushed with excess water and/or by gentle mechanical action (e.g., brushing).

The dental bleaching devices can be worn for as little as a few minutes or as long as several hours. By way of example, not limitation, a typical bleaching session of fast duration may last from about 10 to about 30 minutes. A bleaching session of intermediate duration may last from about 30 minutes to about 2 hours. A bleaching session of long duration, including professional or overnight bleaching while a person is sleeping, may last from about 2 hours to about 12 hours.

Bleaching sessions may be repeated as many times as needed to obtain a desired degree of treatment. A clinical whitening effect has been observed after only 1-3 bleaching sessions. A typical bleaching regimen will preferably include 1-20 bleaching sessions, more preferably 2-15 bleaching sessions, and most preferably 3-10 bleaching sessions.

For convenience of use, multiple dental bleaching devices may be packaged together and sold as a kit. In one embodiment, the number of dental bleaching devices provided with each kit may equal the number of sessions that represent a prescribed bleaching regimen.

To efficiently utilize the space within a kit package, multiple dental bleaching devices can be stacked or interested together. The dental bleaching devices can be sealed collectively or individually as desired.

IV. Examples

Examples of dental bleaching compositions and barrier layers that may be used are disclosed in U.S. patent application Ser. No. 10/790,446, filed Feb. 19, 2004 and titled ORAL TREATMENT DEVICES THAT INCLUDE A THIN, FLEXIBLE BARRIER LAYER AND AN ENDOSKELETON TREATMENT OR ADHESIVE COMPOSITION, hereby incorporated by reference with respect to examples of barrier layers and dental bleaching compositions.

### EXAMPLE 1

A protective coating was formed by dissolving 1 part (by weight) PEROXYDONE K-90 in 9 parts ethanol (by weight) so as to form a 10% solution. The solution was sprayed onto several dental bleaching compositions (both gels and substantially solid compositions), each sample of dental bleaching composition being supported on a barrier layer. The solution was allowed to dry, after which it was observed that the solution had formed a soft shell protective coating over the dental bleaching composition.

### EXAMPLE 2

A hypothetical protective coating is formed by dissolving 2 parts (by weight) PEROXYDONE K-30 in 8 parts water (by weight) so as to form a 20% solution. The solution is sprayed onto several dental bleaching compositions (both gels and substantially solid compositions), each sample of dental bleaching composition being supported on a barrier layer. The solution is allowed to dry, after which it is observed that the solution has formed a soft shell protective coating over the dental bleaching composition.

### EXAMPLE 3

A hypothetical protective coating is formed by dissolving 3 parts (by weight) PEROXYDONE K-30 in 7 parts glycerol (by weight) so as to form a 30% solution. The solution is sprayed onto several dental bleaching compositions (both gels and substantially solid compositions), each sample of dental bleaching composition being supported on a barrier layer. The solution is allowed to dry, after which it is observed that the solution has formed a soft shell protective coating over the dental bleaching composition.

### EXAMPLE 4

A hypothetical protective coating is formed by providing PEROXYDONE K-30 in a white dry powder form. The dry powder is dusted over dental bleaching compositions (both gels and substantially solid compositions), forming a protective coating that adheres to the dental bleaching composition.

### EXAMPLE 5

A hypothetical protective coating is formed by providing PEROXYDONE XL-10 in a white dry powder form. The dry powder is dusted over dental bleaching compositions (both gels and substantially solid compositions), forming a protective coating that adheres to the dental bleaching composition.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A dental bleaching material, comprising:
a dental bleaching composition, said bleaching composition including a dental bleaching agent and a thickening agent; and
a protective coating on a surface of the dental bleaching composition, said protective coating including a complex of polyvinyl pyrrolidone and hydrogen peroxide.

2. A dental bleaching material as recited in claim 1, said dental bleaching agent comprising at least one of aqueous hydrogen peroxide, carbamide peroxide, metal perborates, metal percarbonates, metal peroxides, metal chlorites and hypochlorites, peroxy acids, and peroxy acid salts.

3. A dental bleaching material as recited in claim 1, said thickening agent comprising at least one of polyvinyl pyrrolidone (PVP), PVP-vinyl acetate copolymers, carboxypolymethylene, polyethylene oxide, polyacrylic acid, polyacrylic acid polymers or copolymers, polyacrylates, polyacrylamides, copolymers of polyacrylic acid and co g polyacrylamide, carboxymethylcellulose, carboxypropylcellulose, cellulosic ethers, polysaccharide gums, or protein.

4. A dental bleaching material as recited in claim 1, said dental bleaching composition comprising a gel.

5. A dental bleaching material as recited in claim 1, said dental bleaching composition comprising a substantially solid composition.

6. A dental bleaching material as recited in claim 1, said protective coating comprising a dry powder adhered to said dental bleaching composition.

7. A dental bleaching material as recited in claim 1, said protective coating comprising a shell adjacent to said dental bleaching composition.

8. A dental bleaching material as recited in claim 1, said protective coating comprising from about 5% to about 30% hydrogen peroxide by weight.

9. A dental bleaching material as recited in claim 1, said protective coating comprising from about 10% to about 25% hydrogen peroxide by weight.

10. A dental bleaching material as recited in claim 1, said protective coating comprising from about 15% to about 22% hydrogen peroxide by weight.

11. A dental bleaching device, comprising:
a moisture resistant barrier layer;
a dental bleaching composition adjacent to said barrier layer, said bleaching composition including a dental bleaching agent and a thickening agent; and
a protective coating on a surface of said dental bleaching composition, said protective coating including a complex of polyvinyl pyrrolidone and hydrogen peroxide.

12. A dental bleaching device as recited in claim 11, said dental bleaching composition comprising a bead of dental bleaching gel adjacent to said barrier layer.

13. A dental bleaching device as recited in claim 12, said bead having a cross-sectional diameter between about 1 mm and about 5 mm.

14. A dental bleaching device as recited in claim 12, said bead having a cross-sectional diameter between about 2 mm and about 4 mm.

15. A dental bleaching device as recited in claim 11, said dental bleaching composition being substantially solid and comprising a continuous layer on an inner surface of said barrier layer.

16. A dental bleaching device as recited in claim 15, said continuous layer of said dental bleaching composition having a thickness from about 0.25 mm to about 2 mm.

17. A dental bleaching device as recited in claim 11, said protective coating also covering at least a portion of said barrier layer.

18. A dental bleaching device as recited in claim 11, the device being contained within a sealed package prior to use.

19. A dental bleaching device as recited in claim 11, wherein said barrier layer is initially in the form of a dental tray.

20. A dental bleaching device as recited in claim 11, wherein said barrier layer is initially substantially flat.

21. A method of manufacturing a dental bleaching device comprising:
providing a moisture resistant barrier layer;
applying a dental bleaching composition adjacent to said barrier layer, said bleaching composition including a dental bleaching agent and a thickening agent; and
applying a protective coating on a surface of said dental bleaching composition, said protective coating including a complex of polyvinyl pyrrolidone and hydrogen peroxide.

22. A method as recited in claim 21, wherein said protective coating is applied as a dry powder.

23. A method as recited in claim 21, wherein said protective coating is applied as a solution of said complex of polyvinyl pyrrolidone and hydrogen peroxide dissolved in a solvent.

24. A method as recited in claim 23, wherein said solvent comprises at least one of water, ethanol, glycerol, or a glycol.

25. A method as recited in claim 23, wherein said protective coating is applied by spraying, dipping, or spreading.

26. A method as recited in claim 23, further comprising allowing said protective coating to dry so as to form a shell.
